(19) United States
(12) Patent Application Publication
(10) Pub. No.: US 2004/0002594 A1
(43) Pub. Date: Jan. 1, 2004

(54) Removal of extraneous substances from biological fluids containing nucleic acids and the recovery of nucleic acids

(76) Inventor: John Krupey, Glen Rock, NJ (US)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/180,053

(22) Filed: Jun. 27, 2002

(51) Int. Cl. C07H 21/04; C12P 19/34

(52) U.S. Cl. 536/25.4

(57) ABSTRACT

A method for removing proteins and unwanted aggregated DNA from biological media containing nucleic acids by subjecting the starting material to a water insoluble complex consisting of ProCipitate™ and protein interspersed with ferric oxide particles to a magnetic force.

### BACKGROUND

The use of fumed metallic oxide particles in nucleic acid purification was previously described in Provisional Patent Application Serial No. 60/164,608, entitled Method for Isolating DNA from Proteinaceous Medium and Kit for Performing Method, filed Nov. 10, 1999, the disclosure of which is incorporated herein by reference in its entirety.

The present invention relates to a means for removing proteins and unwanted aggregated DNA from biological media containing desired nucleic acids, by subjecting the starting material to a water insoluble complex consisting of ProCipitate™-protein, or aggregated DNA and protein, interspersed with ferric oxide particles and to a magnetic force; or by interspersing heavy metal oxides such as bismuth oxychloride into the ProCipitate™-protein-aggregated DNA complex and allowing the resulting aggregate to settle under unit gravity.

Hawkins in U.S. Pat. No. 5,705,628 describes a method for separating nucleic acids using magnetic micro-particles. The method as described involves many steps and is expensive. First the magnetic particles must be chemically derivatized to permit nucleic acid attachment. Secondly, the binding conditions are very stringent since they require different iterations of salt and polyethylene glycol. In contradistinction to this procedure, the method employed in the present invention uses underivatized magnetic particles and is not constrained by solvent composition and ionic strength.

Nucleic acids are polymeric acids. In addition to having large numbers of nucleotides and ribose moieties, they possess a plurality of negatively charged phosphate groups. Because of their strong negative charge they should bind tightly to a positively charged fumed metallic oxide surface. It has been demonstrated (Kurnmert R., and Strum W., International Journal of Colloid and Interface Science, 75(2) 373, 1980) that organic molecules with molecular masses smaller than 200 daltons and with the functional groups carboxylic, phenolic —OH or an amino group which can form covalent bonds with the structural metal, bind to the fumed aluminum oxide surface. The compounds that were employed in these studies were phthalic acid, benzoic acid, salicylic acid and catechol. Since the primary focus and objective is the binding of polymeric acids to the oxide surface, very little is to be gained from the studies which employ monomeric molecules.

In general the binding of a polyelectrolyte (e.g. DNA) to a surface containing multiple permanent charges of opposite sign is energetically more favorable than the binding of a single isolated monomeric unit (e.g. a deoxy ribonucleoside triphosphate) to the same surface. The simultaneous presence of multiple interactions when the polyelectrolyte and surface are brought together may produce cooperativity between them, and together they might be much stronger than might be expected from the sum of their individual bond strengths.

In the case of single interactions involving the monomeric molecule and an oppositely charged surface, the single interactions are mutually exclusive or non-cooperative and hence the resulting bonds are relatively weak as compared to those between the polymer and the surface.

Boom et al, U.S. Pat. No. 5,234,809 discloses a method for adsorbing nucleic acids onto silica particles in the presence of chaotropic agents. The silica-nucleic acid complex is then washed with organic solvents to prevent desorption of the nucleic acid from the solid phase. The nucleic acid is then eluted from silica using a mild buffer. There are fundamental differences in the chemistry and physical properties of silica and the fumed metallic oxides of the present invention.

Silica is an oxide of the element silicon. Silicon has properties between metals and non-metals and is called a metalloid. Metallic oxides, such as titanium oxide, are an oxide of metals such as titanium. A metal is a substance having a characteristic luster, malleability and high electrical conductivity, that is, metals readily loose electrons to form positive ions.

A metal can be thought of as an array of nuclei immersed in a sea of electrons; some of the electrons present roam through the array of nuclei and acid and act as an all prevailing electrostatic glue. This is not the case with metalloids (silicon) where the electrons are less promiscuous and have a lesser tendency to wander about. All the atoms of metalloids are held together by a network of electron pair bonds. Substances with this type of structure are referred to as “network covalent solids”. The entire crystal, in effect consists of one huge molecule.

When fumed titanium oxide of the present invention is placed in contact with water, its surface acquires a permanent positive charge. When this positively charged matrix is placed into contact with an aqueous solution of nucleic acid in either pure water, chaotropic salts or non-chaotropic salts (kosmotropes), a strong ionic bond is formed between the positively charged metallic surface and the negatively charged phosphate groups of the nucleic acid. The resulting nucleic acid-fumed titanium oxide complex is stable and cannot be dissociated by treatment with either pure water, alcohol, chaotropic ions or kosmotropic ions under neutral conditions. Dissociation is promoted by treatment with mild alkali.

When silica particles are placed in contact with water they do not acquire a permanent positive charge. Silica particles are mildly acid. Based on the experiments of Boom et al U.S. Pat. No. 5,234,809, it appears that the interactive forces between the silica particles are weak in comparison to the strong electrostatic force that exists between the fumed metallic oxide and the nucleic acid since washing of the complex with pure water or neutral salt solutions tend to release significant amounts of nucleic acid from the surface. As a result of this property, Boom uses organic solvents to wash off extraneous proteins that are co-adsorbed onto the particles. Treating the nucleic acid-silica complex with an aqueous organic solvent to remove contaminating protein might be counterproductive, particularly if the protein is insoluble in that solvent composition.

In order to release significant amounts of DNA from the nucleohistone complex of mammalian cells, the cells are treated with a solution containing a chaotrope. The accepted definition of a chaotrope or chaotropic ion is a substance or anion which is least effective as a protein precipitant, and promotes unfolding, extension, and dissociation (Dandliker, W. B and de Saussure, V. A. in The Chemistry of Biosurfaces, Ed. M. L. Hair, Marcel Dekker, New York, 1971, p18). Examples of chaotropic anions are guanidine thiocyanate and potassium iodide.

At the opposite extremes are the kosmotropic ions. These substances are most effective as protein precipitants and lead to folding, coiling, and association. The helical content of the protein is thereby increased as a result of this treatment. Examples of kosmotropes are sodium chloride and sodium sulfate.

The process of protein destabilization is carried out in the presence of large amounts of chaotropes (3 molar to 10 molar for guanidine thiocyanate). At these concentrations, the extremely chaotic solution conditions overcome the molecular forces and cause destabilization of proteins. Boom et al employed a 10 molar solution of guanidine thiocyanate to displace the DNA from the starting material while a 3 molar solution of the same reagent was employed for dissociation purposes in the fumed metallic oxide procedure.

There is, however, a clear difference between the two methods with regard to the concentration of chaotrope that is employed during the adsorption process. The chaotrope requirements for the adsorption process of Boom, et al, U.S. Pat. No. 5,234,809, are very stringent in that high concentrations of this reagent must be maintained to permit the adsorption of DNA to the silica particles.

In contrast, the chaotrope requirements for adsorption to fumed metallic oxide surfaces are far less stringent, since the binding of DNA to this surface can occur at either high concentrations of chaotrope (5M) or at much lower concentrations of this reagent (0.01M) with equal efficiency.

U.S. Pat. No. 5,057,426 discloses a method for separating long chain nucleic acids comprising fixing the nucleic acids onto a porous matrix, washing the porous matrix to separate the other substances from the long chain nucleic acids, and removing the fixed long chain nucleic acids from the porous matrix. The porous matrix is a material for chromatography having been modified with respect to its surface, and the material is based on a member selected from the group consisting of silica gel, diatomite, aluminum oxide, titanium oxide, hydroxylapatite, dextran, agarose, acrylamide, polystyrene, polyvinyl alcohol or other organic polymers, and derivatives or copolymers thereof.

U.S. Pat. No. 5,470,463 relates to modified porous solid supports and processes for the preparation and use of same. In particular, passivated porous mineral oxide supports are disclosed which are characterized by a reversible high sorptive capacity substantially unaccompanied by non-specific adsorption of or interaction with biomolecules. Passivation is achieved by use of a passivation mixture comprising a main monomer, a passivating monomer and a crosslinking agent, which mixture upon polymerization results in the substantial elimination of the undesirable non-specific interaction with biomolecules.

U.S. Pat. No. 5,599,667 discloses the use of polycationic solid supports in the purification of nucleic acids from solutions containing contaminants. The nucleic acids non-covalently bind to the support without significant binding of contaminants permitting their separation from the contaminants. The bound nucleic acids can be recovered from the support. Also described is the use of the supports as a means to separate polynucleotides and hybrids thereof with a nucleotide probe from unhybridized probe. Assays for target nucleotide sequences are described which employ this separation procedure.

U.S. Pat. No. 5,635,405 discloses an aqueous colloidal dispersion for diagnostic or immunodiagnostic tests, comprising non-polymer nuclei surrounded by a hydrophilic copolymer that contains functional groups, a method for the detection of a specifically binding substance or immunochemically active component in a test fluid, and test kit containing the aqueous colloidal dispersion.

U.S. Pat. No. 5,705,628 discloses a method of separating polynucleotides, such as DNA, RNA and PNA, from a solution containing polynucleotides by reversibly and non-specifically binding the polynucleotides to a solid surface, such as a magnetic microparticle, having a functional group-coated surface is disclosed. The salt and polyalkylene glycol concentration of the solution is adjusted to levels which result in polynucleotide binding to the magnetic microparticles. The magnetic microparticles with bound polynucleotides are separated from the solution and the polynucleotides are eluted from the magnetic microparticles.

There is a need in the art for improved methods for isolating DNA. The present invention overcomes prior art deficiencies in methods of isolating DNA.

### SUMMARY OF THE INVENTION

The present invention provides a means for removing proteins and unwanted aggregated DNA from biological media containing nucleic acids by subjecting the starting material of specimen to a water insoluble complex containing (1) a water insoluble protein bridging network polyelectrolyte having an affinity to aggregate proteins present in biological media while leaving DNA intact in a supernatant (for example, ProCipitate™) and (2) protein interspersed with (3) ferric oxide particles and then subjecting the resulting material to a magnetic force. Alternatively, aggregated DNA interspersed with ferric oxide particles are subjected to a magnetic force. The clear supernatants are recovered and analyzed for nucleic acid.

In another embodiment the invention provides a means for removing proteins and aggregated DNA from biological media containing nucleic acids by reacting a complex of the aforementioned water insoluble protein bridging network polyelectrolyte (for example, ProCipitate™) and protein with heavy metal oxides (e.g. bismuth oxychloride) or by interacting the aggregated DNA with heavy metal oxides, and allowing the respective complexes to settle under unit gravity. The clear supernatants are recovered and analyzed for nucleic acids.

The invention also provides a means for binding fumed metallic oxides to ferric oxide particles. The object is to enable the dissociation of the DNA or RNA from the fumed metallic oxide-Fe₃O₄ complex under mild alkali conditions in a magnetic field.

In an alternative embodiment the invention provides a method for removing proteins and aggregated DNA from biological specimens and removing the desired nucleic acids comprising contacting a specimen including nucleic acids to a water insoluble complex consisting of the aforementioned water insoluble protein bridging network polyelectrolyte (for example, ProCipitate™) and protein interspersed with ferric oxide particles to form a mixture; or contacting a specimen including nucleic acids to a water insoluble complex comprising the aforementioned water insoluble protein bridging network polyelectrolyte (e.g., ProCipitate™) aggregated DNA and protein interspersed with ferric oxide particles; or contacting a specimen including nucleic acids to a water insoluble complex comprising aggregated DNA and protein interspersed with ferric oxide; and applying a magnetic force to said mixture.

In still another embodiment the invention provides a method for removing proteins and aggregated DNA from biological specimens and recovering the desired nucleic acids comprising, contacting a specimen including nucleic acids to a water insoluble complex of the aforementioned water insoluble protein bridging network polyelectrolyte (for example, ProCipitate™) and protein interspersed with a heavy metal oxide such as bismuth oxychloride to form a mixture; or contacting a specimen including nucleic acids to a water insoluble complex comprising the aforementioned water insoluble protein bridging network polyelectrolyte (e.g., ProCipitate™) aggregated DNA and protein interspersed with bismuth oxychloride instead of ferric oxide; and allowing the complex to settle under unit gravity.

The above and other objects of the invention will become readily apparent to those of skill in the relevant art from the following detailed description and figures, wherein only the preferred embodiments of the invention are shown and described, simply by way of illustration of the best mode of carrying out the invention. As is readily recognized the invention is capable of modifications within the skill of the relevant art without departing from the spirit and scope of the invention.

### BRIEF DESCRIPTION OF THE FIGURES

FIG. 1 shows electrophoretic patterns of DNA isolated using magnetized ferric oxide particles Lane 1=Control genomic DNA; Lane 2=DNA isolated from whole blood; Lane 3=BAC DNA from a strain of E. coli; and Lane 4=Plasmid DNA from a strain of E. coli.

FIG. 2 shows electrophoretic patterns of DNA isolated using bismuth oxychloride; Lane 1=Control genomic DNA; Lane 2=DNA isolated from whole blood; Lane 3=BAC DNA from a strain of E. coli; and Lane 4=Plasmid DNA from a strain of E. coli.

FIG. 3 shows a schematic of a method of isolation of DNA from whole blood using ferric oxide.

FIG. 4 shows a schematic of a method of isolation of DNA from whole blood using bismuth oxychloride.

FIG. 5 shows a schematic of a method of isolation of plasmid DNA using ferric oxide.

FIG. 6 shows a schematic of a method of isolation of plasmid DNA using bismuth oxychloride.

FIG. 7 shows the preparation of protein bridging network polyelectrolytes (PBNP).

FIG. 8 shows a proposed mechanism for the aggregation of proteins by protein bridging network polyelectrolytes (PBNP) and their desorbtion.

### DESCRIPTION OF THE INVENTION

The present invention relates to a means for removing proteins and unwanted aggregated DNA from biological media containing nucleic acids by subjecting starting material of a water insoluble complex containing the water insoluble protein bridging network polyelectrolyte (e.g., ProCipitate™) and protein interspersed with ferric oxide particles to a magnetic force.

The method of the invention can advantageously be used, for example, in high throughput diagnostics, molecular bioinformatics, nucleic acid isolation and characterization.

Advantages of the present invention include:

a) The methods described obviate the necessity for centrifugation or filtration process steps;

b) The methods described are user friendly, cost effective and amenable for process automation; and

c) The methods described permit the rapid removal of contaminating nucleic acids in the downstream processing.

### EXAMPLE 1

Isolation of DNA from Whole Blood using Ferric Oxide Particles

Fifty microliters of whole mouse blood in a suitable container are treated with a 3M solution of guanidine thiocyanate containing O.1M ethylene diamine-tetraacetate (EDTA).

Two hundred and fifty microliters of the water insoluble, protein-aggregating agent, ProCipitate™ are then added.

One hundred microliters of a 10% aqueous suspension of finely divided ferric oxide (Fe₃0₄) particles are then added and the tubes are mixed. At this stage of the procedure, the ferric oxide particles become interspersed within the protein ProCipitate™ aggregate thus making this configuration amenable to the action of a magnetic force.

The aggregates are drawn to the inner wall of the tube using a magnet. The clear supernatant containing the nucleic acids is withdrawn from the tube by pipette.

The nucleic acid is isolated according to the procedure described in provisional Patent Application Serial No. 60/164,608, entitled Method for Isolating DNA from Proteinaceous Medium and Kit for Performing Method, filed Nov. 10, 1999, the contents of which are incorporated herein by reference in their entirety. An example of this procedure follows and is present in Example 5 below. In this procedure, one volume of whole blood is treated with two volumes of a chaotropic agent such as 3M guanidine thiocyanate in a buffer, for example about 100 mM sodium acetate pH 7.0. After standing at room temperature for about 15 minutes a suspension of the protein precipitator ProCipitate™ (manufactured by LigoChem Inc., Fairfield N.J.) is then added to precipitate the protein. The composition of ProCipitate™ is disclosed in U.S. Pat. Nos. 5,294,681; 5,453,493 and 5,534,597, and U.S. application Ser. No. 08/676,668 (now allowed), and the disclosures of each of these three listed U.S. patents and the listed application are incorporated herein by reference in their entireties.

The tubes are then centrifuged at 10,000×g for 15 minutes, and the supernatant recovered, 1.5 volumes of Titanium Oxide P-25 is then added. The resulting aggregate consisting of DNA and metallic oxide is allowed to settle under unit gravity. After settling the supernatant is removed by aspiration and the settled complex is washed with three washings using deionized water. The tubes are then centrifuged at about 1000×g for 30 seconds. The supernatant is discarded and 0.02M sodium hydroxide is added to the tube. The tubes are then vortexed, followed by centrifugation at about 10,000×g for 5 minutes. The supernatants are then removed, neutralized with a 0.1M Tris HCl solution and analyzed for DNA by spectrophotometric absorption at 260 and 280 nm. One ml of whole blood contains approximately 40 to 50 micrograms of DNA. This quantity translates into about one absorbance unit (AU) at 260 nm and 0.8 AU at 280 ma. The DNA specimens are also subjected to agarose gel electrophoresis in which the DNA bands were identified by ethidium bromide staining.

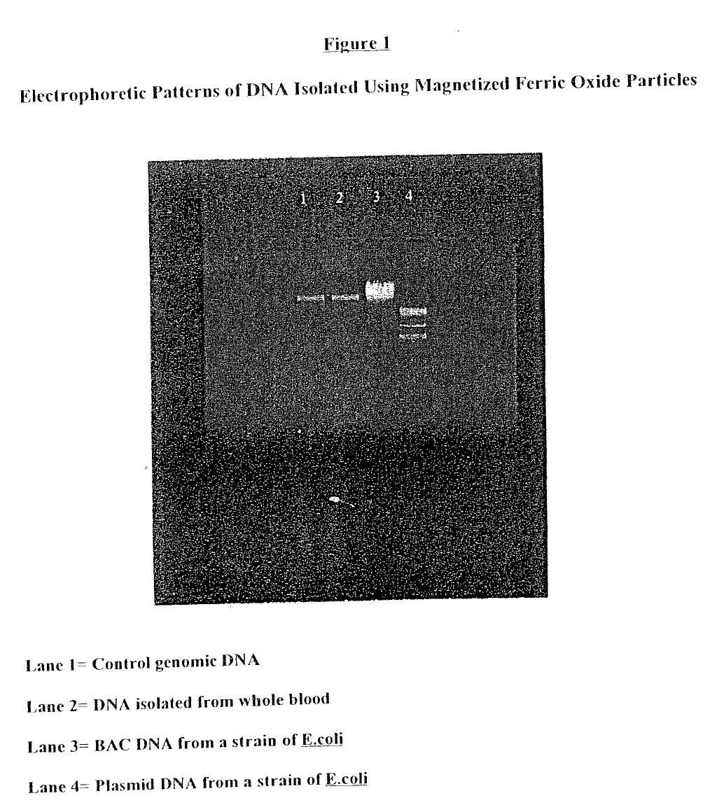

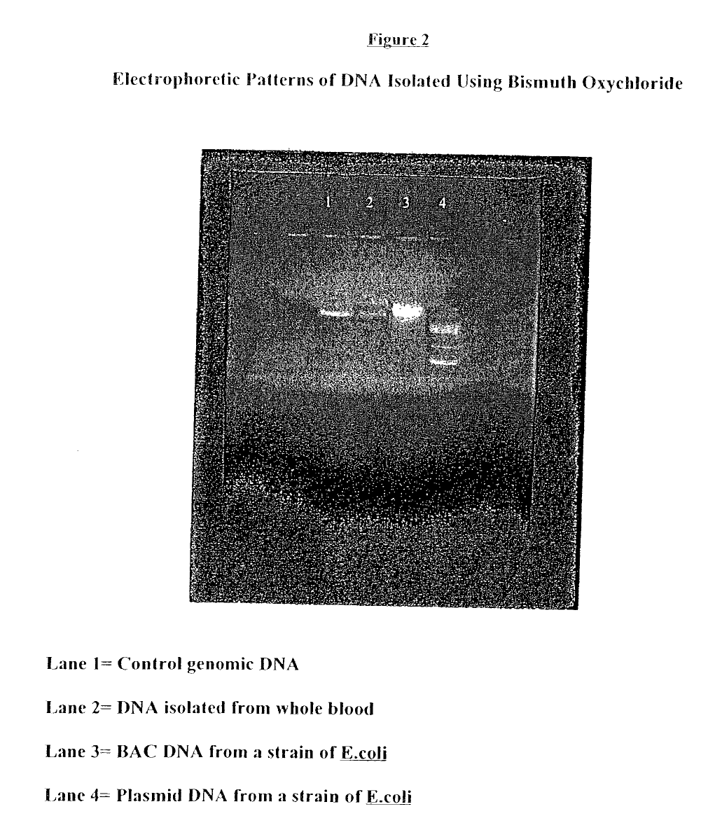

The electrophoretic profile of the genomic DNA isolated is shown in the enclosed FIG. 1.

### EXAMPLE 2

The Isolation of Plasmid DNA from Bacterial Lysates using Ferric Oxide Particles

Two hundred fifty microliters (LB) of an overnight culture containing 10⁹ E. coli plasmid containing cells per ml were prepared.

The cells were centrifuged and the supernatants were discarded.

The cells were then dispersed in 20 ul of Tris buffer pH 8.0 containing RNAse.

Twenty microliters of 1.0% sodium dodecyl sulphate (SDS) were added.

One hundred microliters of a 10% suspension of ferric oxide was then added and the tubes were mixed. This was followed by the addition of 20 microliters of a 5M potassium acetate solution. Ferric oxide becomes interspersed within the mucinous framework of the aggregated DNA. ProCipitate™ can also be employed in this process to further remove extraneous substances along with the ferric oxide particles.

Plasmid DNA is recovered in the clear supernatant after subjecting the aggregate to a magnetic field.

The plasmid DNA is further purified by the procedure described in provisional Patent Application Serial No. 60/164,608, entitled Method for Isolating DNA from Proteinaceous Medium and Kit for Performing Method, filed Nov. 10, 1999.

The electrophoretic profile of the plasmid DNA isolated is shown in the enclosed FIG. 1.

### EXAMPLE 3

Isolation of Plasmids from Bacteria

Recovery

In small-scale production process, growth is carried out in multi-well micro-titer plates to a high cell density (OD₆₀₀=30-100). The cells are then recovered by centrifugation. Cells are then re-suspended and concentrated in a buffer appropriate for the following step and designed to disrupt cells and release the plasmid. This buffer usually contains agents that disrupt the non-covalent bonds between lipids/and or proteins; for instance, ethylene diamine tetra-acetic acid (EDTA) is often used as a chelating agent. The removal of divalent cations (e.g., Ca²⁺ and Mg²⁺) from the cell wall, outer membrane (in Gram-negative bacteria) and plasma membrane destabilizing their structure, facilitating lysis.

Lysis

Bacterial cell lysis is traditionally carried out under alkaline conditions in the presence of the ionic surfactant sodium dodecyl sulfate (SDS) (Ref Birnboim H. C. and Doly J. Nucleic Acids Research 7: 1513-1523. 1979). At this stage of processing, aggregated chromosomal DNA, protein containing bound SDS molecules (SDS-protein) and free plasmid DNA are released into the surrounding milieu, which results in an increase in the viscosity of the solution.

The next step in the lysis procedure is the addition of a high-salt neutralization solution usually potassium acetate which neutralizes the negative charges on the SDS-protein as well as other components and promotes the formation of aggregates of chromosomal DNA and SDS protein complexes. The plasmid DNA remains in solution after this treatment.

The aggregates of chromosomal DNA and SDS-protein are highly gelatinous and in most cases will occlude the membrane filter when one attempts to recover the desired plasmid DNA using a preliminary filtration step. This situation presents an intractable problem particularly in robotic systems where the isolation of plasmid DNA in a multi-microtiter well format must be rapid and efficient.

We have found that this problem may be circumvented by first binding ferric oxide (Fe₃0₄) to the aggregate driving the Fe₃O₄-chromosomal DNA-SDS protein complex to the bottom of the container by applying a magnetic force and recovering the plasmid DNA in the clear supernatant.

Alternatively, a heavy metal oxide such as bismuth oxy-chloride (BiOCl) can be employed in place of Fe₃O₄ as an aggregate binding agent. The BiOCl-chromosomal DNA protein-SDS complex that is formed settles rapidly under unit gravity and thus permits the recovery of the desired plasmid DNA in the clear supernatant.

The plasmids, that are recovered, using either protocol may be purified further by using the traditional alcohol precipitation method or the LigoChem fumed metallic oxide (DNAble) method.

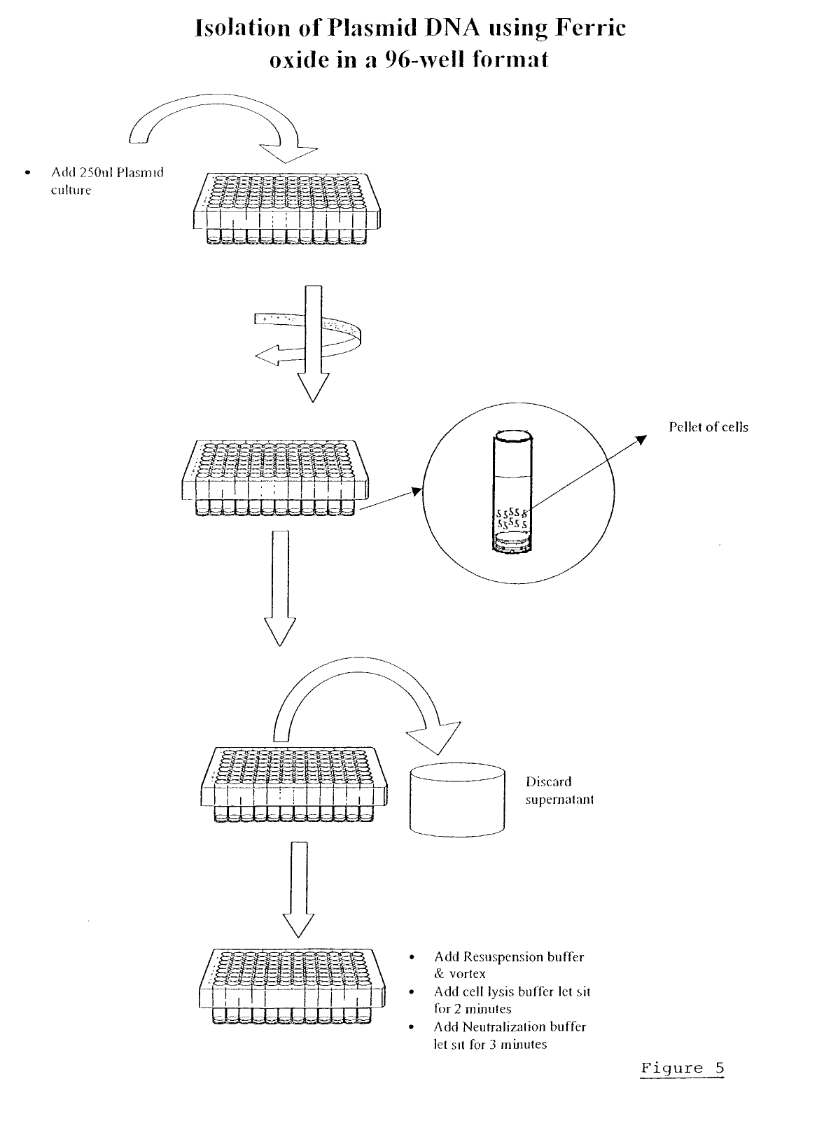

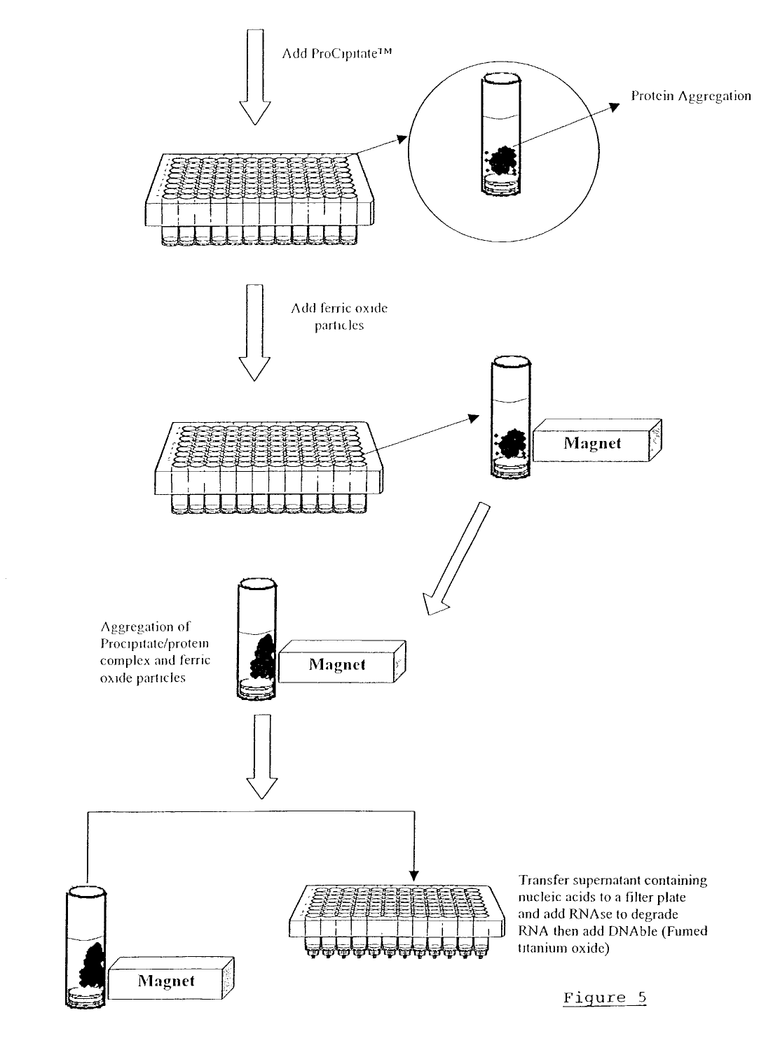

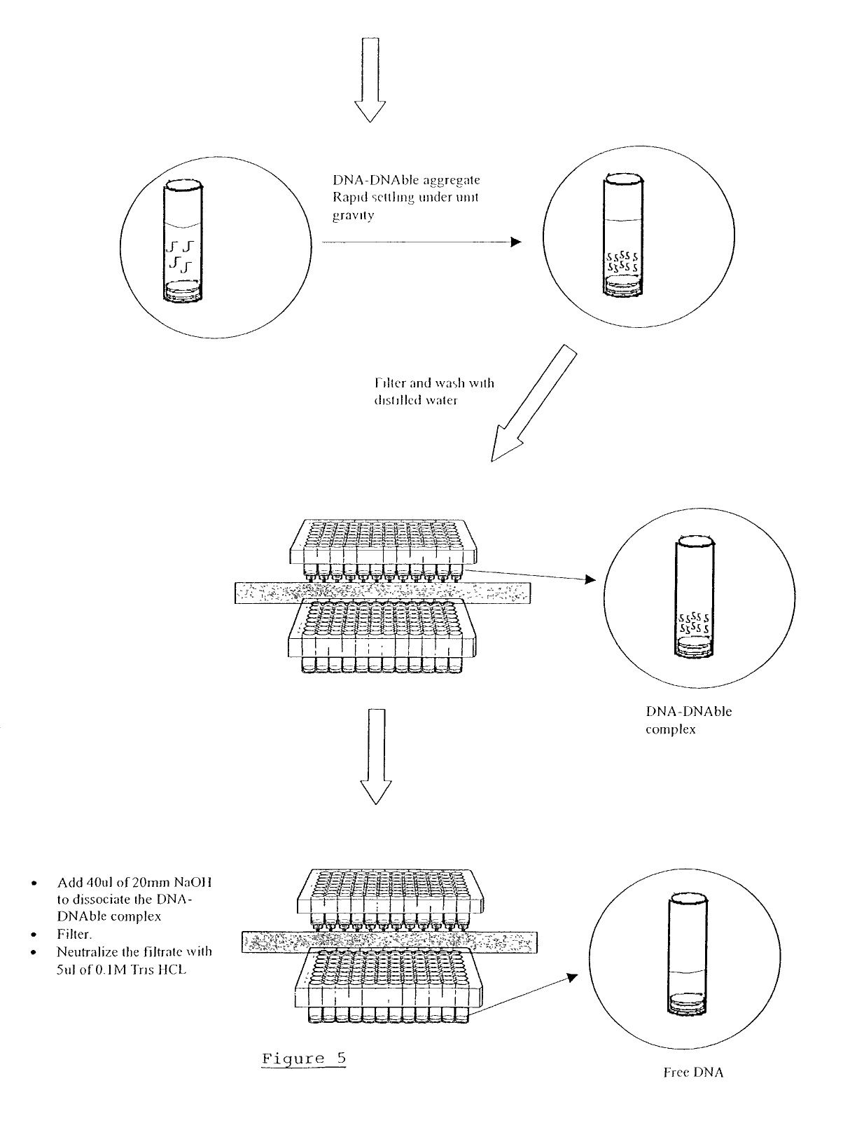

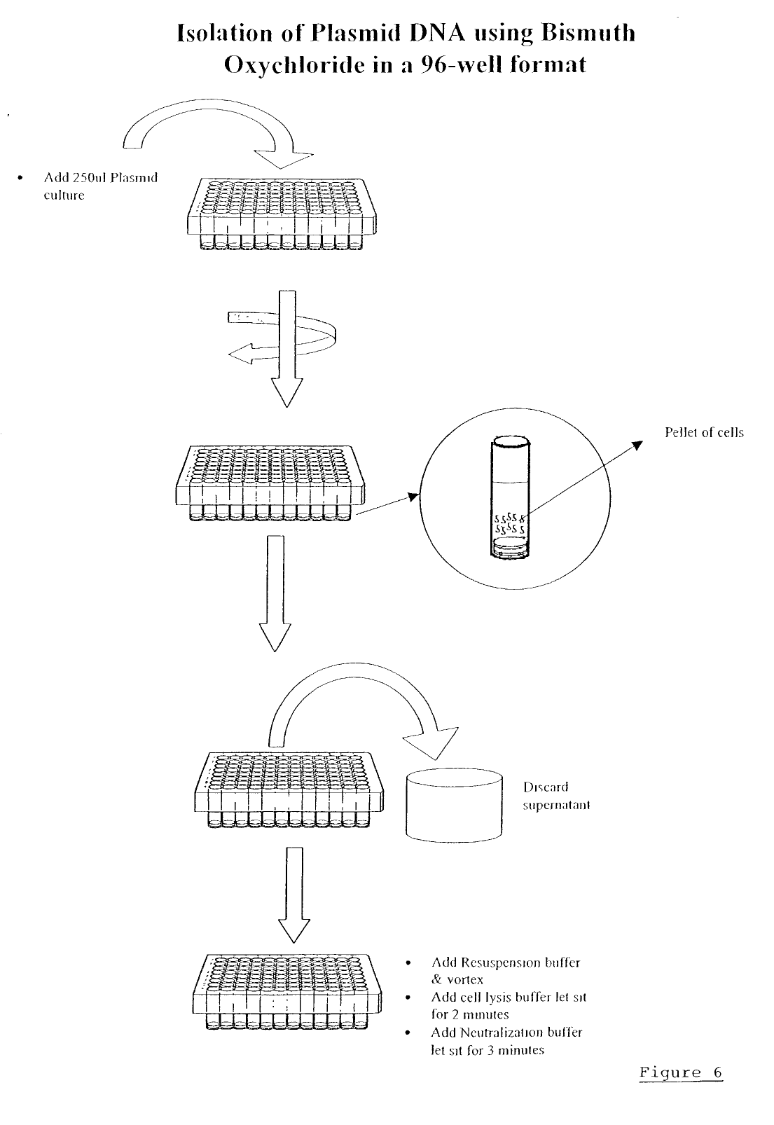

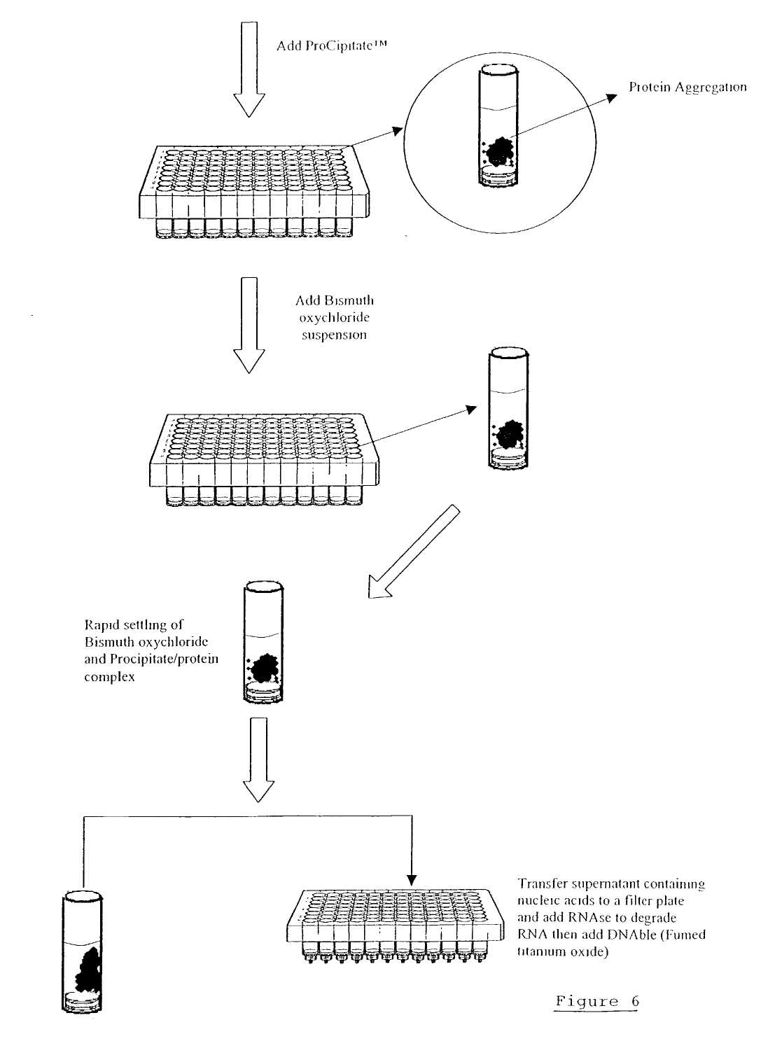

ProCipitate™, a protein aggregating reagent may be added to the neutralized cell lysate to effect the removal of residual proteins, followed by the addition of either Fe₃O₄ or BiOCl. However, it cannot be stated with absolute certainty at this time as to whether this treatment is absolutely necessary in all cases to obtain amplifiable and sequenceable plasmid and BAC DNA. Since bacterial cultures show a marked variation in protein content it can only be surmised that high protein containing cultures require ProCipitate™ pretreatment while those containing lesser amounts of protein do not. FIG. 5 shows a schematic of a method of isolation of plasmid DNA using ferric oxide. FIG. 6 shows a schematic of a method of isolation of plasmid DNA using bismuth oxychloride.

### EXAMPLE 4

Isolation of BAC DNA from Bacterial Lysates using Ferric Oxide Particles

The method employed for the isolation of BAC DNA was essentially the same employed for the isolation of plasmid DNA except that 2.0 ml of bacterial culture was employed instead of 250 microliters.

The electrophoretic profiles of the BAC DNA, isolated is shown in the enclosed FIG. 1.

### EXAMPLE 5

Isolation of DNA from Whole Blood and Bacterial Lysate using Heavy Metal Oxides

Genomic DNA, plasmid DNA, and BAC DNA were isolated from the respective sources by treating lysates with a 10.0% suspension of bismuth oxychloride (BiOCl) and allowing the resulting complexes consisting of extraneous substances and BiOCl to settle under unit gravity in the absence of a magnetic field. The DNA was recovered and purified as described in the Disclosure Document No. 456808. The electrophoretic profiles of the DNA, isolated are shown in FIG. 2.

### EXAMPLE 6

Nucleic Acid Isolation

In this procedure, one volume of whole blood is treated with two volumes of a chaotropic agent such as 3M guanidine thiocyanate in a buffer, say, 100 mM sodium acetate pH 7.0. After standing at room temperature for 15 minutes a suspension of the protein precipitator ProCipitate™ (manufactured by LigoChem Inc., Fairfield N.J.) is then added to precipitate the protein. The composition of ProCipitate™ is disclosed in U.S. Pat. Nos. 5,294,681; 5,453,493; and 5,534,597, and U.S. application Ser. No. 08/676,668 (now allowed) incorporated herein by reference in their entireties.

The tubes are then centrifuged at 10,000×g for 15 minutes, and the supernatant recovered, 1.5 volumes of Titanium Oxide P-25 is then added. The resulting aggregate consisting of DNA and metallic oxide is allowed to settle under unit gravity. After settling the supernatant is removed by aspiration and the settled complex is washed with three washings using deionized water. The tubes are then centrifuged at 1000×g for 30 seconds. The supernatant is discarded and 0.02M sodium hydroxide is added to the tube. The tubes are then vortexed, followed by centrifugation at, say, 10,000×g for 5 minutes. The supernatants are then removed, neutralized with a 0.1M Tris HCl solution and analyzed for DNA by spectrophotometric absorption at 260 and 280 nm. One ml of whole blood contains approximately 40 to 50 micrograms of DNA. This quantity translates into about one absorbance unit (AU) at 260 nm and 0.8 AU at 280 nm. The DNA specimens are also subjected to agarose gel electrophoresis in which the DNA bands were identified by ethidium bromide staining.

In another version of this procedure one volume of blood is treated with two volumes of 3M guanidine thiocyanate in 100 mM sodium acetate (EDTA is not present). The mixture is then heated at 65 degrees Celsius for 10 minutes. After standing at room temperature for 5 minutes, a suspension of ProCipitate™ is added to precipitate the protein. The supernatant is recovered by centrifugation and this DNA containing solution is processed and analyzed for DNA as described above.

Alternatively, one volume of whole blood is treated with three volumes of a 1.0% w/v of sodium dodecyl sulfate (SDS) in a buffer, say, 10 mM solution of Tris buffer and 100 mM EDTA pH 8.0. After remaining at room temperature for 15 minutes, 3 volumes of a 3M solution of potassium acetate is added to neutralize the SDS and to precipitate the hemoglobin that is present. The tubes are then centrifuged and the supernatant is recovered. 1.5 ml of Titanium Oxide P-25 suspension is then added. The aggregate is allowed to settle under unit gravity. After settling the supernatant is discarded and the DNA-metallic oxide complex is washed with three washings of deionized water. The tubes were then centrifuged at 1000×g for 30 seconds and the supernatant discarded. Dissociation of the complex was accomplished by the same method that was used in the ProCipitate™ guanidine thiocyanate procedure.

In view of the above, the methods of the present invention can advantageously be used for:

(a) General screening of blood samples in a 96 well-automated microtiter plate format for genetic aberrations.

(b) Forensic medicine, molecular bioinformatics.

(c) In an automated system for the isolation of bacterial and viral constructs for genomic sequencing.

(d) Non-invasive diagnostics-capture and quantification of DNA in saliva-capture and quantification of small quantities of DNA present in large volumes of urine.

(e) Removal of contaminating nucleic acids in the downstream processing of recombinant proteins.

The procedure is in the DNA recovery protocol. The DNA is routinely eluted from the fumed titanium oxide particles by mild alkali treatment. Under these conditions the metallic oxide particles will not sediment so the suspension must be filtered or centrifuged to recover the DNA. An ideal configuration consists of an alkali stable complex of fumed metallic oxides and ferric oxide that binds DNA, and is attracted by a magnet under mild alkali conditions. Under such conditions, the DNA appears in the clear supernatant after magnetization.

A complex consisting of fumed titanium oxide and ferric oxide has been prepared in the presence of polyethylene glycol. This complex binds DNA and is attracted by a magnet. However, this complex is unstable under mild alkali conditions; dissociating into free fumed metallic oxide, free ferric oxide and free polyethylene glycol.

### EXAMPLE 7

Isolation of DNA from Whole Blood

In order to obtain a DNA specimen which is suitable for amplification and sequencing from a highly proteinaceous medium such as whole blood, the proteins must first be removed. Most available methods are either arduous or painstaking and may require the use of organic solvents to effect protein removal.

A reagent, ProCipitate™, has been shown to be effective in aggregating large quantities of protein present in biological media while leaving the DNA intact in the supernatant. This reagent is currently employed in the isolation of DNA from whole blood.

ProCipitate™ belongs to a class of water insoluble network polyelectrolytes that selectively bind and aggregate proteins and viruses (Krupey, J., U.S. Pat. No. 5,294,681 Mar. 15, 1994; Krupey J., U.S. Pat. No. 5,453,493 Sep. 26, 1995; Krupey, J., U.S. Pat. No. 5,534,597 Jul. 9, 1996; Krupey, J.; Smith, A D.; Arnold, E; and Donnell; U.S. Pat. No. 5,658,779 Aug. 19, 1997; Krupey, J. U.S. Pat. No. 5,976,382, Nov. 2, 1999, the contents of each of these U.S. patents being incorporated herein by reference in their entireties).

These reagents have been collectively named protein bridging network polyelectrolytes (PBNP).

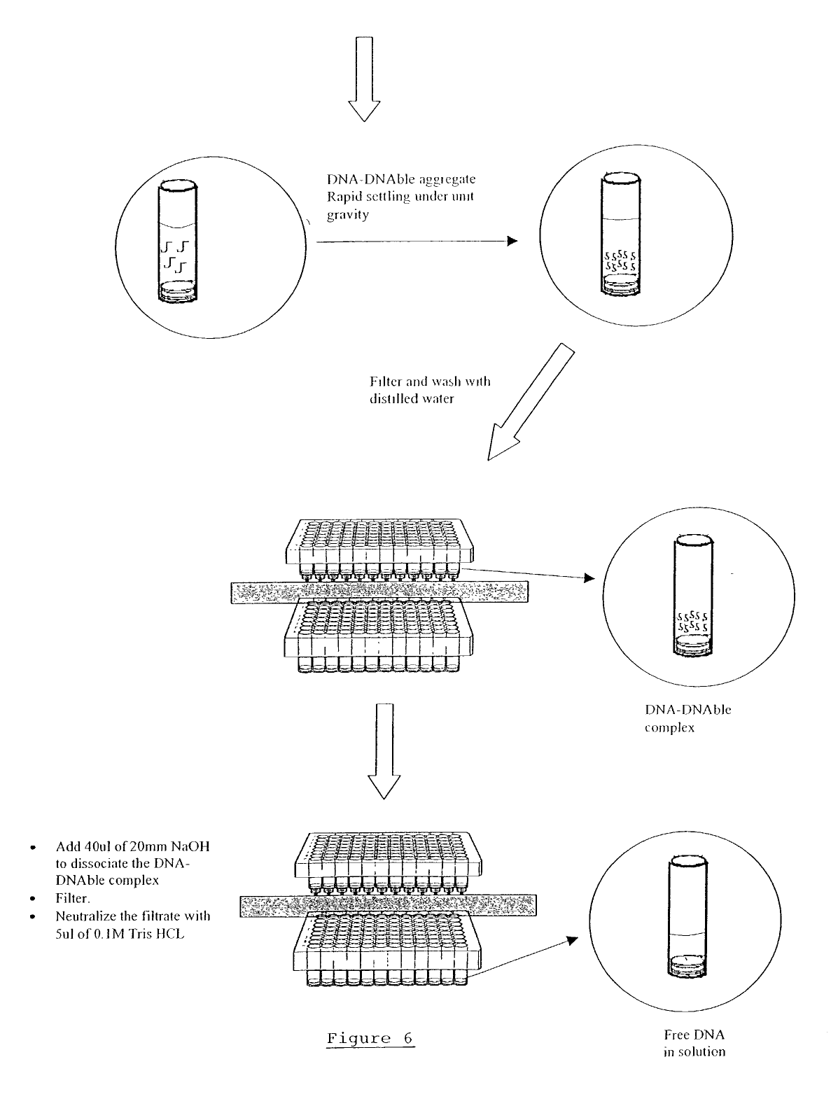

The skeletal framework of these polymers is generated by reacting a polymeric maleic anhydride co-polymer with an aliphatic diamine to yield a network of polycarboxylic acid chains covalently cross-linked by diamide bonds (FIG. 7.) By controlling the chemistry, architecture and charge properties of these structures, it was possible to produce a polymeric configuration that can specifically bind and aggregate the molecules of interest.

These network polyelectrolytes have been engineered to have a slight imbalance between the Coulombic attractive forces that cause the network to shrink or to collapse and the repulsive forces that cause the network to expand. The polymer is routinely employed with only a fraction of its total number of carboxylic acid groups in the ionized form. Therefore, the repulsive interaction predominates, but only marginally. In addition to slightly expanding the network, the repulsive force increases the chemical potential of the polyelectrolyte, a condition that favors its binding to oppositely charged groups present on proteins.

In order for the network polyelectrolytes to bind and aggregate molecules, they must be sufficiently flexible. Since the polymers are chemically cross-linked, parts of the polymer chain could be entwined. This would result in decreased flexibility, since rotations about single bonds would be restricted. The only remaining way in which the chains can flex is by deformation of the bond angles by periodic lengthening and shortening of covalent bonds. Consequently, all the deformations add up along the chain and result in some degree of flexibility.

The number and distribution of charged and polar to apolar residues at the surface of protein molecules is the primary aspect that determines their solubility in a given solvent. Although apolar or hydrophobic groups tend to be concentrated around the interior of protein molecules, some hydrophobic side chains remain exposed to water at the molecular surface or in crevices. These hydrophobic clusters in contact with an aqueous environment cause an ordering of water molecules into extensive hydrogen bonded configurations effectively “freezing” them about the side chains. One important aspect of this phenomenon is a reduction in the number of permitted configurations, equivalent to a decrease in entropy.

Assuming there are no other considerations, the presence of clusters of hydrophobic residues on the surface will favor protein-protein interaction and the formation of multi-unit complexes. Thus in those proteins possessing quaternary structure, the sub units appear to be held together by interactions between hydrophobic clusters on their surfaces.

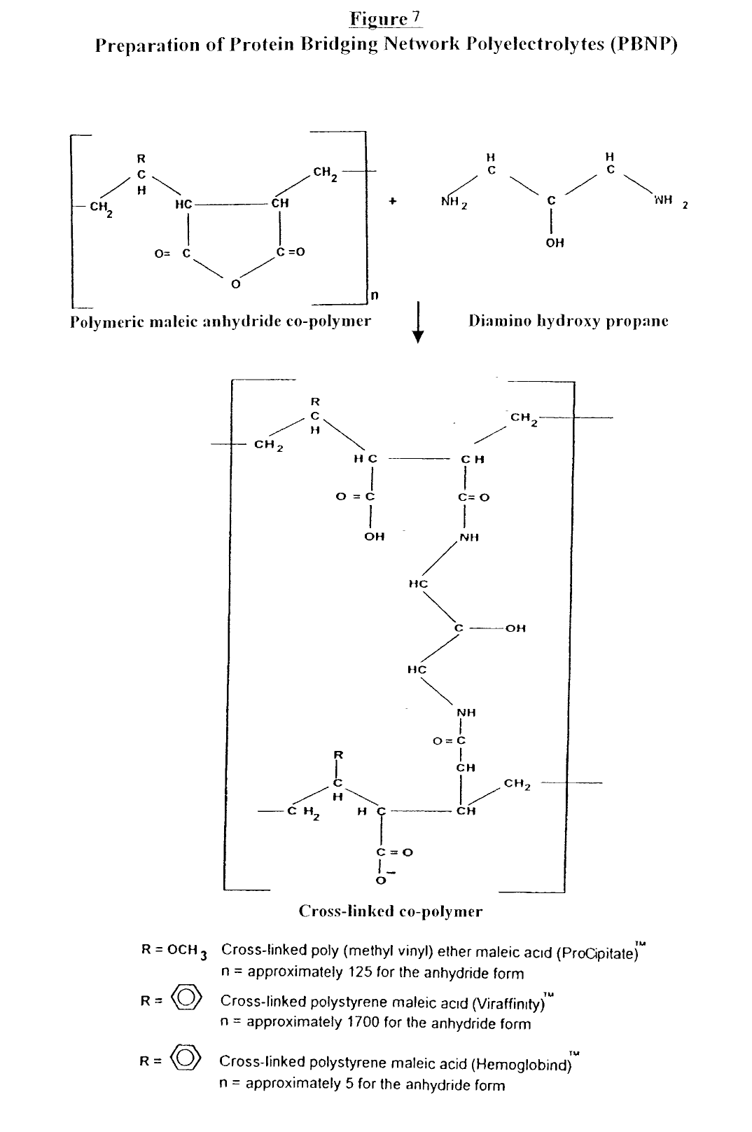

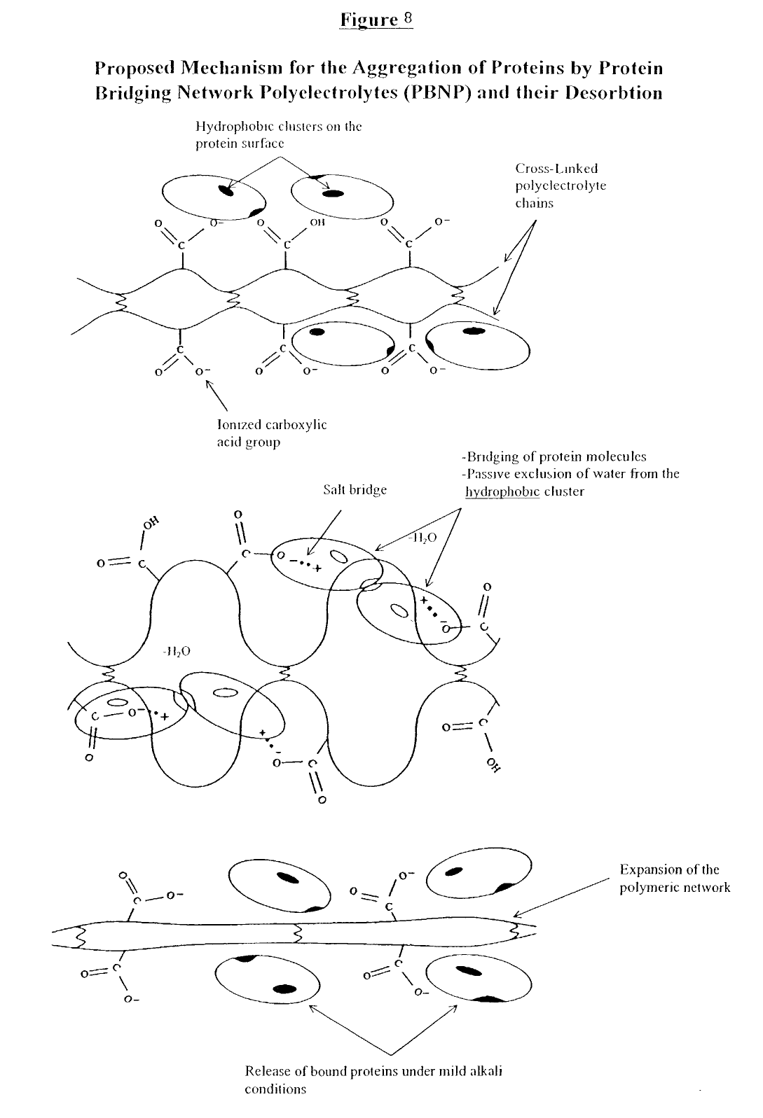

It is postulated that ordered water structures can be disrupted by treatment with the cross-linked polyelectrolytes (FIG. 8). These reagents are flexible and can effectively bridge two or more protein molecules by salt bridges formed by the carboxylate ions of the polymer and the ionized amino groups of the protein. As a consequence of this interaction, the apolar moieties of the individual molecules are brought into close proximity; water is passively excluded and the proteins aggregate while being electrostatically bound to the polymer. The end result is a net increase in the entropy of the system.

A fundamental aspect of protein bridging by the network polyelectrolyte is the energy change that occurs in the course of binding. The polyelectrolyte is initially in a high energy (unfavorable) state because of the strong electrostatic repulsions between the negatively charged monomeric units. When these groups interact with the oppositely charged amino groups on the protein, energy is released and salt bridges may be formed between the carboxylate ions and positively charged amino groups. The release of immobilized water surrounding the ionic groups provides an additional driving force for salt bridge formation. The complex that results then collapses to a state of lower energy which is favorable.

The protein can be dissociated from the complex under mild alkaline conditions (pH 8.5-9.5). Under these conditions, the undissociated carboxylic acid groups on the polymer ionize and strongly repel each other. As a result of this repulsive interaction, the polymeric network expands and the protein is released. A number of cross-linked polycarboxylic acids with different chemistries, architectures, electrical properties and functional binding properties have been prepared.

ProCipitate™ which was prepared from a linear high molecular mass (≧20 kD) aliphatic polyanhydride, was found to be functional in the 3-6.2 pH range. This reagent has a high protein aggregating capacity and is capable of aggregating at least an equivalent weight of either serum albumin or immunoglobulin G originally present in a physiological medium.

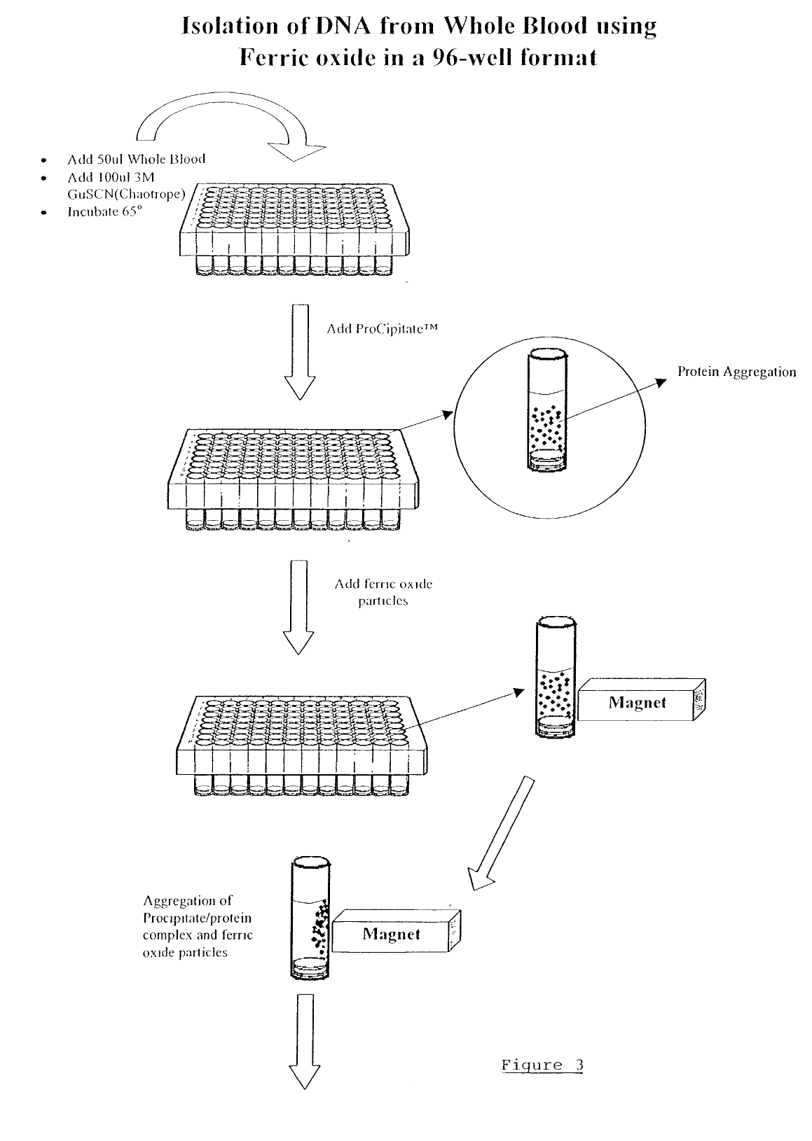

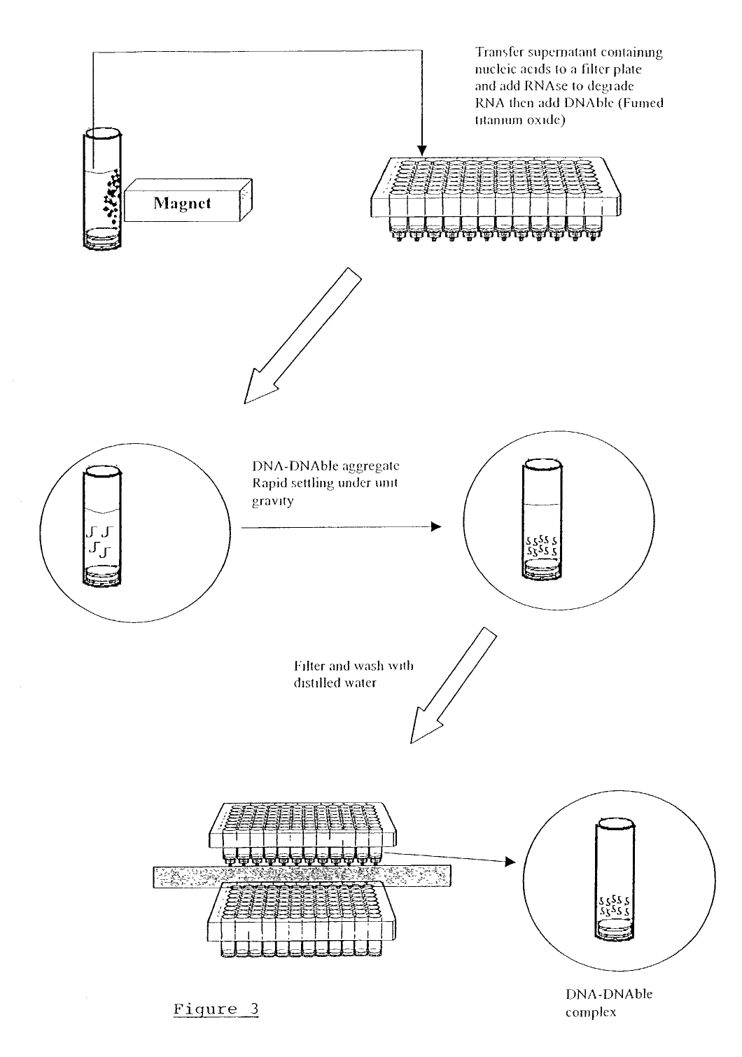

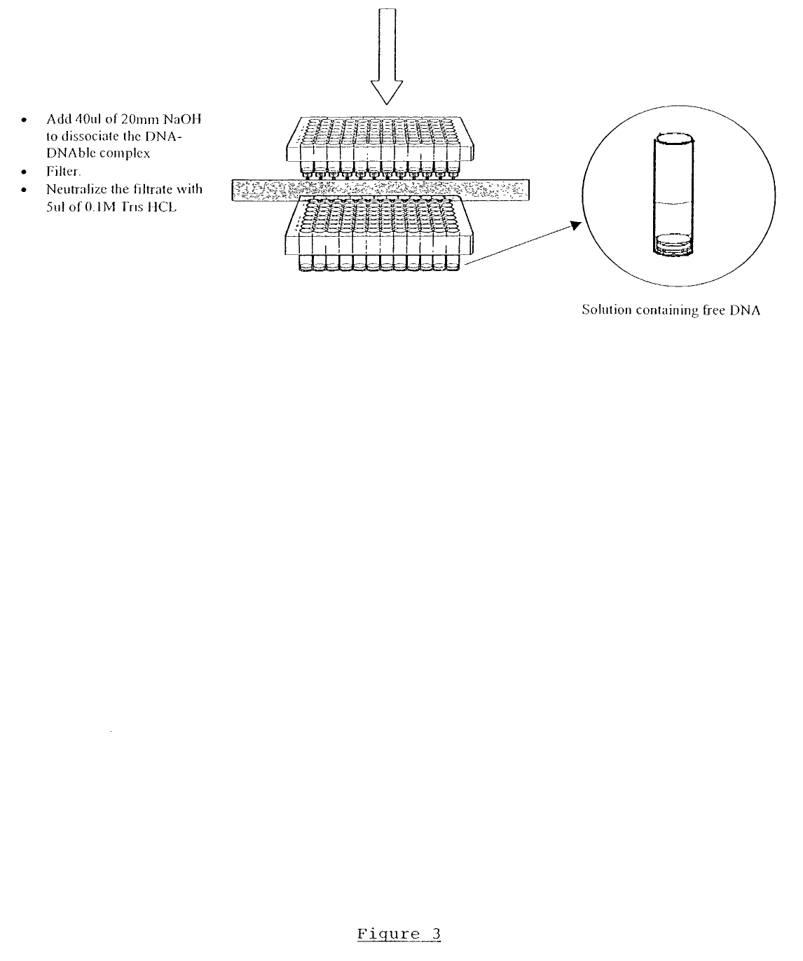

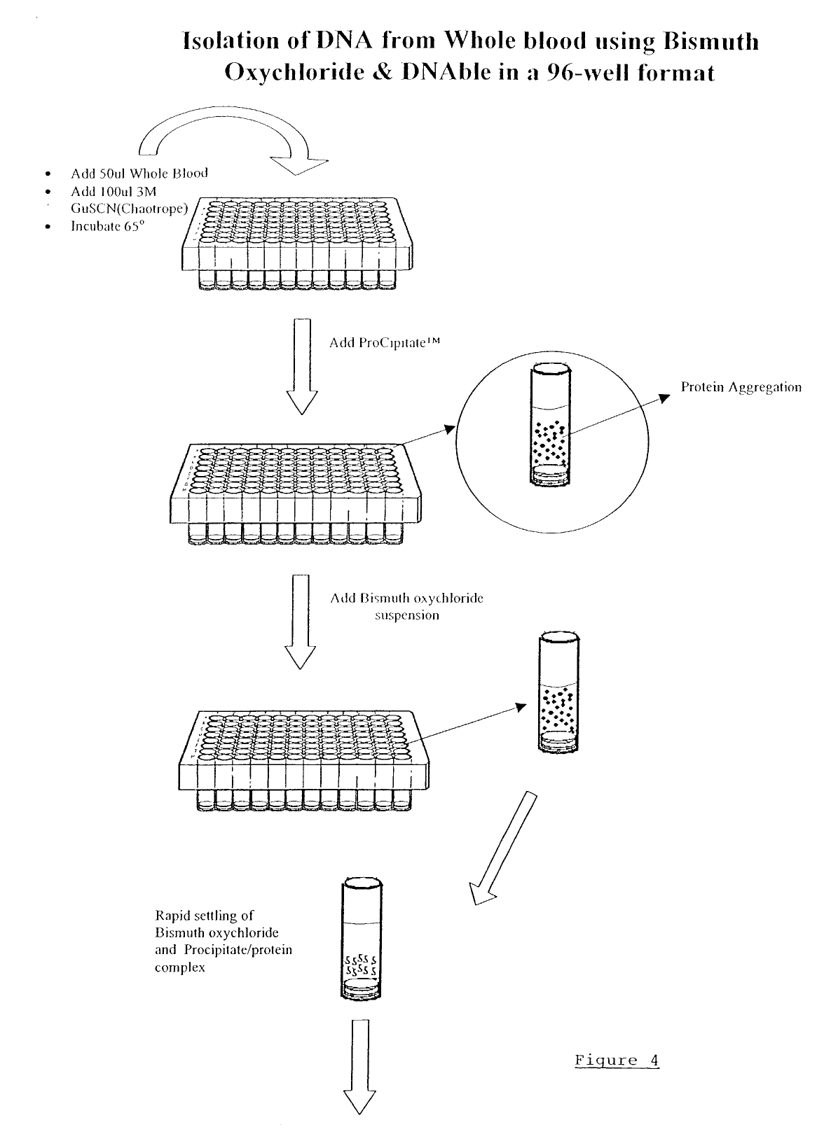

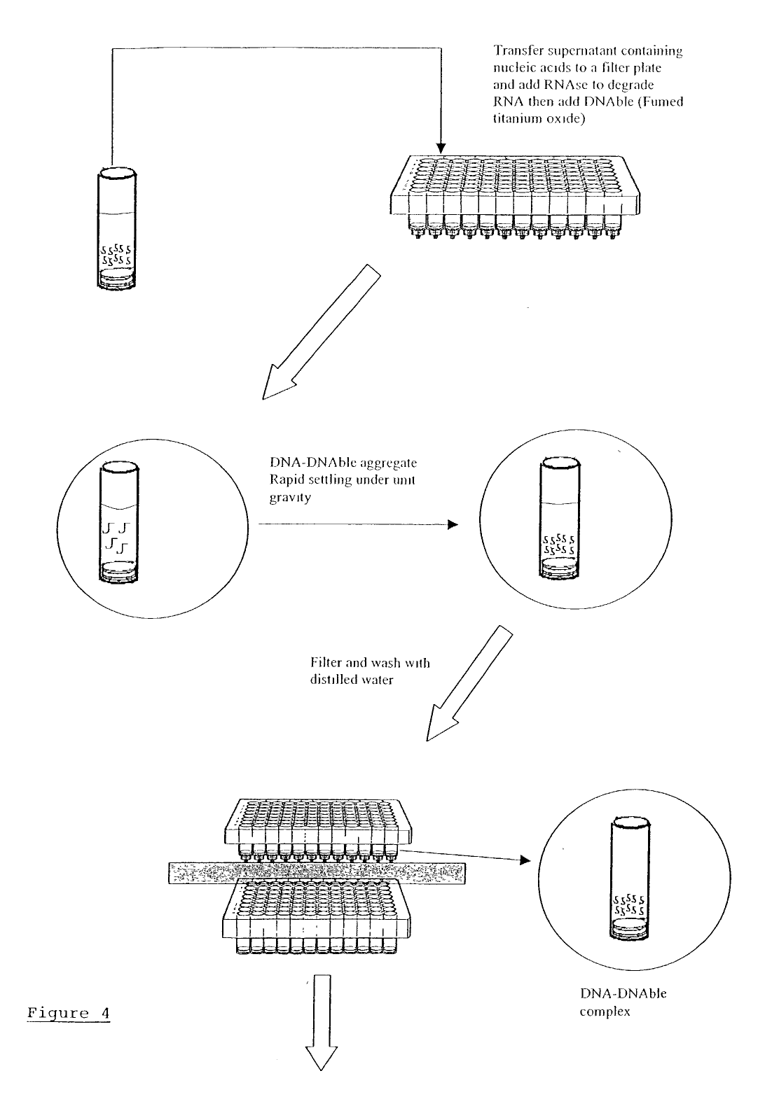

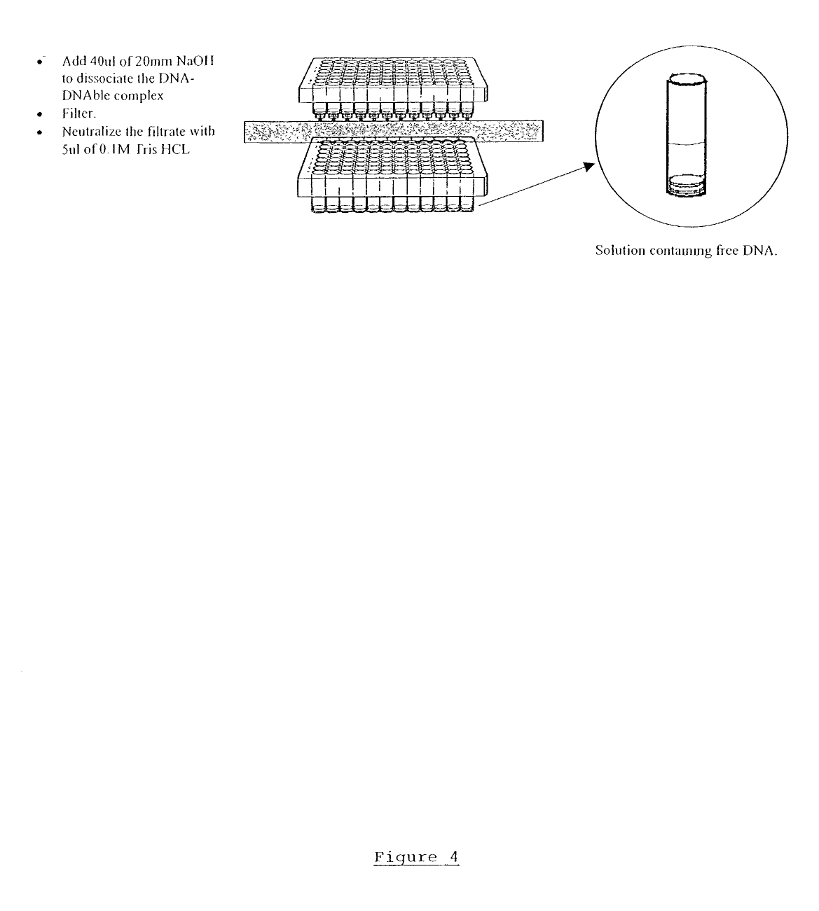

Two network polyelectrolytes with similar chemistries, but with different geometric profiles, were also evaluated. These configurations were prepared from styrene maleic anhydride co-polymers, which differed in their respective molecular masses. Viraffinity™, a virus capture reagent, was derived from a styrene maleic anhydride co-polymer with an average molecular mass of 350 kD. HemogloBind™, a reagent with a high affinity from hemoglobin was prepared from a styrene maleic anhydride co-polymer with an average molecular mass of 1.0 kD. The functional pH range of both types of polyelectrolytes was found to be between 5.5 and 7.5. FIG. 3 shows a schematic of a method of isolation of DNA from whole blood using ferric oxide. FIG. 4 shows a schematic of a method of isolation of DNA from whole blood using bismuth oxychloride.

The purpose of the above description and examples is to illustrate some embodiments of the present invention without implying any limitation. It will be apparent to those of skill in the art that various modifications and variations may be made to the composition and method of the present invention without departing from the spirit or scope of the invention. All patents and publications cited herein are incorporated herein by reference in their entireties.

1. A method for removing proteins and unwanted aggregated DNA from biological specimen containing nucleic acids, comprising:
(A) contacting a specimen including nucleic acids with a water insoluble complex comprising (1) a water insoluble protein bridging network polyelectrolyte having an affinity to aggregate protein present in biological media while leaving DNA intact in a supernatant and (2) protein interspersed with (3) ferric oxide particles to form a mixture, and
(B) applying a magnetic force to said mixture.

2. The method of claim 1, wherein said nucleic acids are selected from the group consisting of DNA and RNA.

3. The method of claim 1, further comprising isolating the nucleic acid from the specimen by
(A) treating the specimen with a chaotropic agent containing a metal chelator, or alternatively heating the specimen in the presence of the chaotropic agent without the chelator being present,
(B) adding a water insoluble protein aggregating agent, said water insoluble protein bridging network polyelectrolyte, and isolating a liquid phase,
(C) treating the liquid phase with an adsorbent consisting of alumina, titania or zirconia generated by flame hydrolysis,
(D) separating the supernatant,
(E) washing the residue with deionized water,
(F) removing deionized water wash and then dissociating the DNA from the fumed alumina, titania, or zirconia by treatment with aqueous alkali borate or phosphate or a metal hydroxide, and
(G) recovering and neutralizing the liquid phase containing DNA.

4. The method of claim 1, wherein the water insoluble protein bridging network polyelectrolyte is ProCipitate™.

5. A method for removing proteins and aggregated DNA from biological specimens and removing the desired nucleic acids, comprising:
contacting a specimen including nucleic acids to a water insoluble complex containing of (1) a water insoluble protein bridging network polyelectrolyte having an affinity to aggregate protein present in biological media while leaving DNA intact in a supernatant and (2) protein interspersed with (3) ferric oxide particles to form a mixture; or
contacting a specimen including nucleic acids to a water insoluble complex comprising said protein network bridging polyelectrolyte aggregated DNA and protein interspersed with ferric oxide particles to form a mixture; or
contacting a specimen including nucleic acids to a water insoluble complex comprising aggregated DNA and protein interspersed with ferric oxide to form a mixture; and
applying a magnetic force to said mixture.

6. The method of claim 5, wherein the water insoluble protein bridging network polyelectrolyte is ProCipitate™.

7. A method for removing proteins and aggregated DNA from biological specimens and recovering the desired nucleic acids, comprising:
contacting a specimen including nucleic acids to a water insoluble complex containing of (1) a water insoluble protein bridging network polyelectrolyte having an affinity to aggregate protein present in biological media while leaving DNA intact in a supernatant and (2) protein interspersed with (3) a heavy metal oxide to form a mixture; or
contacting a specimen including nucleic acids to a water insoluble complex comprising said protein network bridging polyelectrolyte aggregated DNA and protein interspersed with bismuth oxychloride; and
allowing the complex to settle under unit gravity.

8. The method of claim 7, wherein said nucleic acids are selected from the group consisting of DNA and RNA.

9. The method of claim 7, wherein the water insoluble protein bridging network polyelectrolyte is ProCipitate™.

10. The method of claim 5, wherein said nucleic acids are selected from the group consisting of DNA and RNA.

11. The method of claim 7 further comprising isolating the desired nucleic acid from the specimen by:
(A) treating the liquid phase with an adsorbent containing of alumina, titania, or zirconia generated by flame hydrolysis,
(B) separating the supernatant,
(C) washing the residue with deionized water,
(D) removing the deionized water wash and then dissociating the DNA from the fumed alumina, titania, or zirconia by treatment with aqueous alkali borate or phosphate or a metal hydroxide, and
(E) recovering and neutralizing the liquid phase containing DNA.

12. The method of claim 5 further comprising isolating the desired nucleic acid from the specimen by:
(A) treating the liquid phase with an adsorbent containing alumina, titania, or zirconia generated by flame hydrolysis,
(B) separating the supernatant,
(C) washing the residue with deionized water,
(D) removing the deionized water wash and then dissociating the DNA from the fumed alumina, titania, or zirconia by treatment with aqueous alkali borate or phosphate or a metal hydroxide, and
(E) recovering and neutralizing the liquid phase containing DNA.

13. The method of claim 2, further comprising isolating the desired nucleic acid from the specimen by precipitating the desired nucleic acid by adding the specimen to an alcohol contained in a vessel equipped with a filtration membrane by removing the alcohol by vacuum or pressure filtration,
drying the membrane containing the nucleic acid by vacuum suction or by applying pressure, and
adding a small volume of water or buffer to the membrane to solubilize the nucleic acid and permit its recovery.

14. The method of claim 1, further comprising isolating the desired nucleic acid from the specimen by precipitating the desired nucleic acid by adding the specimen to an alcohol contained in a vessel equipped with a filtration membrane by removing the alcohol by vacuum or pressure filtration,
drying the membrane containing the nucleic acid by vacuum suction or by applying pressure, and
adding a small volume of water or buffer to the membrane to solubilize the nucleic acid and permit its recovery.

15. A method for removing proteins and unwanted aggregated DNA from a biological specimen containing nucleic acids, comprising:
(A) contacting a specimen including nucleic acids with a water insoluble complex comprising aggregated DNA and protein interspersed with ferric oxide particles or bismuth oxychloride, to form a mixture, and
(B) applying a magnetic force to said mixture.
